(19) United States
(12) Patent Application Publication
(10) Pub. No.: US 2022/0062025 A1
(43) Pub. Date: Mar. 3, 2022

(54) DEVICE FOR IN-FLIGHT URINE ELIMINATION

(71) Applicant: Angele Innovations, LLC, San Marcos, CA (US)

(72) Inventors: Janice M. Shields, San Marcos, CA (US); Christian T. Darabant, Chester Springs, PA (US)

(21) Appl. No.: 17/409,453

(22) Filed: Aug. 23, 2021

Related U.S. Application Data
(60) Provisional application No. 63/070,193, filed on Aug. 25, 2020.

(51) Int. Cl. A61F 5/451; A61F 5/44; B64D 10/00

(52) U.S. Cl. CPC A61F 5/451; A61F 5/4401; A61F 2005/4402; A61F 5/4408; B64D 10/00; A61F 5/4405

(57) ABSTRACT

A disposable garment incorporates a system for collecting fluid waste from a user, i.e., urine, while the user is unable to visit a bathroom for a lengthy time duration. A collection chamber in the system receives urine flow from the user, and the collection chamber connects with a bladder which receives the urine via a one-way Tesla-type tube which prevents backflow during fluid transfer. Also incorporated into the garment is a pair of saddlebags, with each bag connected to a respective portion of the bladder for receiving and holding urine therefrom. For this purpose, each saddlebag contains a liquid absorbing material impregnated with a drying agent. Operationally, each bag is designed to conform against a leg of the user for the expected time duration. At the end of the time duration, the garment and bags are disposed of.

This application claims the benefit of U.S. Provisional Patent Application Ser. No. 63/070,193, filed Aug. 25, 2020. The entire contents of Application Ser. No. 63/070,193 are hereby incorporated by reference herein.

### FIELD OF THE INVENTION

The present invention pertains to enclosed systems and operational methods for managing and controlling fluid transfer and collection for subsequent disposal. In particular, the present invention pertains to athletic style garments that incorporate fluid transfer and collection components, which can be worn by users during prolonged periods of time, under circumstances where the user has no access to a location where fluid waste products can be properly eliminated and disposed. The present invention is particularly, but not exclusively useful for the collection and disposal of human waste products such as urine and menstrual discharge.

### BACKGROUND OF THE INVENTION

When a person's workplace is small and confining, and the person must work there for a prolonged period of time, an important concern is the requirement to accommodate the person's need to discretely, functionally and comfortably relieve oneself of fluid waste products, e.g., urine and/or menstrual discharge. The situation is particularly problematic when the prolonged time period must remain uninterrupted for as much as sixteen hours or more. For example, pilots of high-performance aircraft who routinely have extensive inflight mission requirements, or extended operational readiness duties, are particularly vulnerable and susceptible. Furthermore, they do not need the distraction caused by an uncomfortable situation.

Factors for concern in designing a self-contained fluid transfer garment include both practical and health considerations. In the event, the paramount concern is for the health and wellbeing of the person. As a practical matter, an apparatus that is incorporated in a fluid transfer garment must necessarily be leak proof. Further, due to the fact the apparatus must engage with the user's body around his/her genitalia, the apparatus must have a conformable engaging component for initially collecting the fluids that is comfortable, and that creates a leak-proof seal with the user's body. Moreover, user acceptance will depend on factors of personal concern. These factors include flexibility, comfort, convenience and optimal muscle flexion and extension for the user.

With the above in mind, it is an object of the present invention to provide a self-contained disposable system for collecting fluid waste from a user over a prolonged time duration that gives the user reliable, safe, and constant assurance of its operation. Still another object of the present invention is to provide a disposable system for collecting fluid waste from a user that is anatomically contoured to properly accommodate both human physiological functions and external factors. Yet another object of the present invention is to provide a disposable system for collecting fluid waste from a user which is easy to use, is simple to manufacture, and is comparatively cost effective.

### SUMMARY OF THE INVENTION

A system and method for collecting fluid (liquid) waste from a user establishes a fluid flow course that begins with a catcheter that engages with the user. For purposes of the present invention, there are two versions of the catcheter. One is a female version, and the other is a male version. They both, however, have certain common characteristics. Specifically, both versions include a soft silicone structure that forms a hollow collection chamber which extends between a first end and a second end. The first end of the respective collection chambers is designed to engage the user with a fluid-tight seal around his/her genitalia. The second end is connected with a one-way valve to prevent backflow into the collection chamber. From there, the systems are essentially identical.

The female version of the catcheter is formed with a cup-like shape wherein the first end is formed as a lip with a groove. A hydrocolloid is placed in the groove to ensure a fluid-tight seal, and a splash guard is located inside the hollow chamber adjacent the lip. Both the lip and the splash guard continue around the periphery of the open first end of the female catcheter. Additionally, faux hairs can be constructed adjacent to the second end of the hollow chamber to reduce fluid turbulence inside the collection chamber.

The male version of the catcheter is essentially a hollow collection chamber with a penis hole at the first end. The area on the outside of the collection chamber, around the penis hole, is contoured for a comfortable engagement with the male user. An anti-flowback ring is affixed inside the collection chamber at its second end.

A three-way stop-cock valve is provided to give the system user a choice between three different operational configurations for the present invention. These configurations are for: 1) external urine collection for immediate independent disposal; 2) urine collection over an extended time duration for subsequent disposal with the entire system; and 3) providing a pneumatic assist for the second configuration.

For the first configuration, the three-way valve connects the catcheter directly with an external collection bulb for a separate, independent disposal of urine. The purpose here is two-fold. First, it can preserve the system for a subsequent use. Second, it can delay a use of the system for later in a mission when the mission may possibly continue to extend for an unknown time duration.

The second configuration of the system serves the purpose for which the system was intended, namely: long term availability for urine relief. In this configuration, the three-way valve interconnects the one-way valve from the catcheter with two one-way Tesla tubes. Each Tesla tube is then connected directly into fluid communication with a respective tubular shaped bladder. Urine is then transferred from the bladder to a collection bag (i.e., a saddlebag) where it is held until the entire system is disposed.

For any of several reasons, urine flow from the catcheter and into the bladder may be slower than desired. In this situation, a third configuration for the three-way valve can be established whereby a pneumatic pressure variation in the bladder is exercised to draw sluggish urine flow toward the bladder.

At the end of the fluid course through the system, when the three-way valve is in its second configuration, urine from the bladder enters a collection bag, hereinafter frequently referred to as a saddlebag. In essence, a saddlebag is a liquid proof bag constructed to hold a layer of a fluid absorbent material, such as compressed sponge, that has been impregnated with a drying agent, e.g., sodium polyacrylate. Further, an upper surface of this layer is formed with a fluid channel complex for dispersing urine across the surface for absorption. A plurality of holes that avoids the fluid channel complex is also created. These holes extend through the layer of absorbent material and the liquid proof bag it is in, A grommet is then inserted through each hole in the layer, and is connected in a liquid tight seal with the bag at the upper and lower surfaces of the layer.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which;

FIG. 1 is a profile view of a user wearing a system for collecting fluid waste;

FIG. 2 is schematic presentation of the separate fluid (liquid/air) flow patterns established by the system of the present invention;

FIG. 3 is a schematic layout of the components employed by the present invention to control liquid flow through the system;

FIG. 4 is a cross section view of a bifurcated bladder as seen along the line 4-4 in FIG. 3, with liquid/air connections in fluid communication with liquid/air sections of the bladder;

FIG. 5A is a presentation of air pressure components for exercising fluid flow in the system;

FIG. 5B is a presentation of fluid (liquid) components for configuring the system to accomplish an immediate, external disposal of liquid from the system;

FIG. 5C is a Tesla valvular conduit configured for use with the present invention;

FIG. 6 shows the functional components of the present invention mounted on a garment for wear by a user;

FIG. 7A is a perspective view of a female catcheter;

FIG. 7B is a perspective view of the female catcheter, with portions broken away for clarity;

FIG. 7C is a perspective view of the female catcheter mounted on a garment for wear by a user;

FIG. 7D is a rear view showing a garment for a female user configured for elimination of solid waste;

FIG. 8A is a perspective view of a male catcheter;

FIG. 8B is a perspective view of the male catcheter, with portions shown in phantom for clarity;

FIG. 8C is a perspective view of the male catcheter mounted on a garment for wear by a user;

FIG. 9 is an exploded view of a saddlebag;

FIG. 10A is a perspective view of an assembled saddlebag;

FIG. 10B is a grommet used for the assembly of a saddlebag; and

FIGS. 11 A-C respectively show a user wearing the innermost garment layer provided to support a catcheter (FIG. 11A); a user wearing a complete complement of components for the fluid flow system [catcheter, Teals tube, bladder, and saddlebag] mounted on the innermost garment layer (FIG. 11B); and a user wearing a complete system, including the outermost garment layer (FIG. 11C).

### DESCRIPTION OF THE PREFERRED EMBODIMENTS

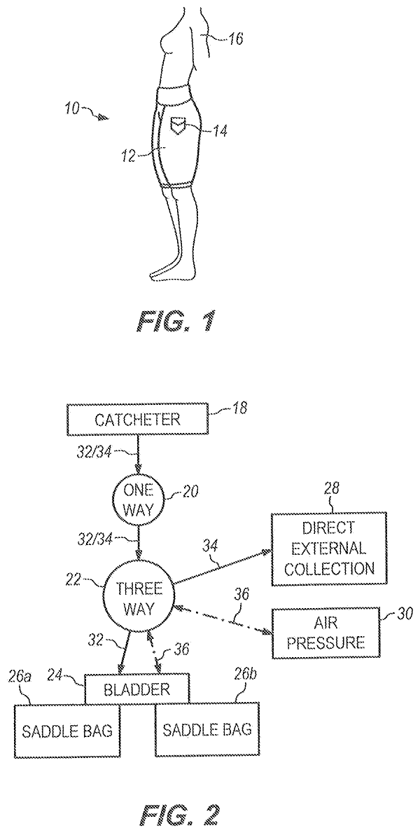

Referring initially to FIG. 1, a system generally designated 10 incorporates components which are mounted inside an outer garment layer 12. As shown, the outer garment layer 12 can include at least one external pocket 14 for holding auxiliary equipment of the system 10, such as a coconut oil wipe (not shown). As intended for the present invention, when the outer garment layer 12 is worn by a user 16 it will extend from the waist to just above the knees. For purposes of the present invention, depending on the operational environment, the system 10 can be under a flight suit or some type of overalls.

Referring now to FIG. 2, essential components of the system 10 are shown arranged for establishing fluid flow paths envisioned for the system 10. Component configurations for two liquid flow paths for the present invention include a catcheter 18, whose purpose is to receive liquid waste, i.e. urine and/or menstrual discharge, from the user 16. Immediately downstream from the catcheter 18 is a one-way valve 20 which presents a backflow into the catcheter 18 of any liquid in the system 10 that is downstream from the one-way valve 20.

Overall fluid flow control within the system 10 of the present invention is provided by a three-way valve 22 that is connected immediately downstream from the one-way valve 20. Specifically, the three-way valve 22 can be manipulated by the user 16 to separately establish either of two different liquid pathways, or one air flow pathway through the system 10. The primary pathway for an operation of the system 10 is a liquid pathway that starts at the catcheter 18 and is directed from the three-way valve 22 into a tube-shaped bladder 24 where the liquid (urine) is further directed for collection into either of two saddlebags 26a or 26b. Another liquid pathway can be established by the user 16 with the three-way valve 22 which provides for direct liquid transfer from the catcheter 18 to an external device 28 for direct collection and disposal. For an operation that is separate from either of the two liquid pathways, an air pathway can be established by the three-way valve 22. Specifically, this air pathway connects the bladder 24 in a direct, two-way air flow with an air pressure pump 30 to assist with an activation of the bladder 24 in low-flow conditions, when needed.

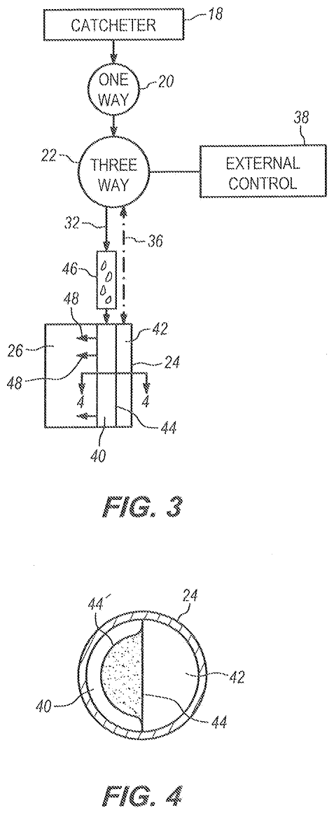

Still referring to FIG. 2, a consolidated statement of the various pathways through the three-way valve is provided. The two liquid pathways 32/34, which are shown as solid lines, extend from the catcheter 18 to the three-way valve 22. At the three-way valve 22, the pathway 32 goes on to the bladder 24, where the liquid urine is then directed into the saddlebags 26a and 26b. The pathway 34, however, is directed from the three-way valve 22 to the external device 28 for collection and disposal. On the other hand, the air pathway 36, shown as a dot-dash line, extends between the air pressure pump 30 and the bladder 24. Importantly, each of the pathways 32, 34 and 36 are individually established by the user 16 manipulating an external control 38 (see FIG. 3).

In FIG. 3, it will be seen that the tube-shaped bladder 24 is lengthwise bifurcated into a fluid holding section 40 and an air pressure section 42. This bifurcation is defined by a flexible membrane 44 that extends along the length of the bladder 24. Preferably, the flexible membrane 44 is made of an expandable elastomeric material. Further, FIG. 3 shows that a Tesla tube 46 is included in the liquid pathway 32 that connects with the fluid holding section 40 of the bladder 24. The purpose of the Tesla tube 46 is to prevent liquid backflow in the system 10 from the bladder 24. FIG. 3 also shows that the air pressure section 42 is in fluid connection with the air pathway 36 and the liquid received into the fluid holding section 40 of the bladder 24 is directed into the saddlebag 26 as indicated by arrows 48.

For this construction, although FIG. 3 shows the pathways 32 and 36 axially aligned with the bladder 24 for disclosure purposes only, it is to be a understood that these pathways 32/36 are preferably connected with the bladder 24 from a lateral, radial direction. Indeed, the preferred point of connection between the pathways 32/36 and the bladder 24 is at the midpoint of the bladder 24.

With reference to FIG. 4 it will be appreciated that an overpressure in the pressure section 42, which is caused by an operation of the pressure pump 30 by the user 16, will inflate the pressure section 42. This will cause the flexible membrane 44 to move into a position indicated for the inflated membrane 44′. In turn, this will decrease the volume of the fluid holding section 40. The purpose is two-fold. For one, an inflation of the pressure section 42 that is caused by an overpressure can help force liquid from the bladder 24 into the saddlebags 26a and 26b. On the other hand, as the overpressure in the pressure section 42 is decreased, more liquid can be drawn from the system 10 into the fluid holding section 40. In each case, the motive is to draw as much liquid into the saddlebags 26a and 26b as possible whenever there may otherwise be a sluggish fluid flow.

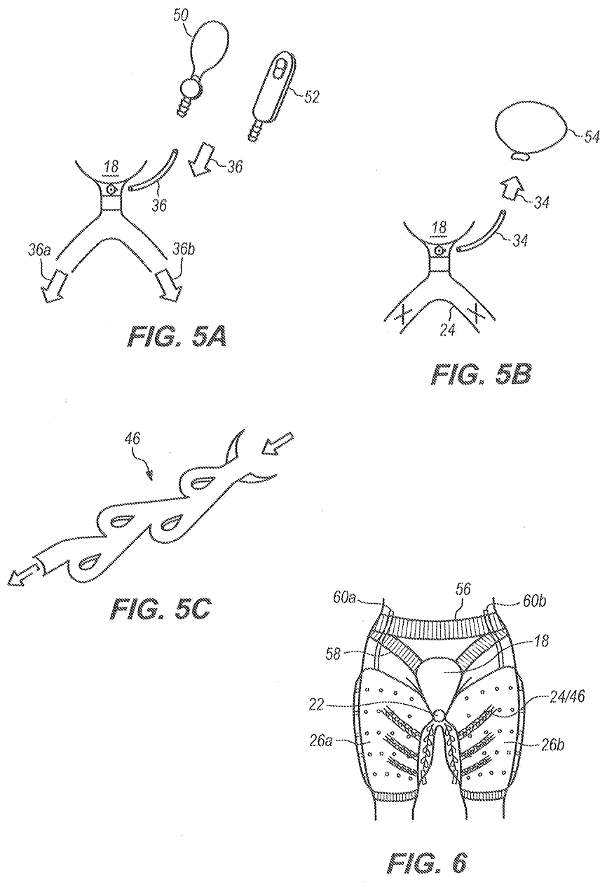

FIG. 5A is a configuration for the system 10 showing components which are specifically intended to pneumatically exercise the system 10 to accelerate and facilitate fluid flow through the system 10. As indicated, the air pressure pump 30 (see FIG. 2) may be either a hand pump 50, of a type typically used with a sphygmomanometer, or it may be a battery-operated electrical pump 52 which is commercially available. FIG. 5B on the other hand, is a configuration for the system 10 showing components which are specifically intended to create a liquid pathway 34 which connects the catcheter 18 in direct fluid communication with an external collection bulb 54. When filled, the collection bulb 54 can be disposed of immediately, or replaced, in a convenient manner. FIG. 5C is a diagrammatic presentation of Tesla valvular tube 46 which can be positioned at various locations on the pathway 32, depending on design requirements. For instance, a Tesla tube 46 can be placed for entry into the fluid holding section 40 of the bladder 24, as shown in FIG. 3 or it can be included with the bladder 24 for direct interaction with the saddlebags 26a and 26b as shown in FIG. 6.

In FIG. 6 the essential components of the system 10 are shown engaged with the user 16. In detail, a waist band 56 and a support band 58 are provided to position and secure the catcheter 18 around the genitalia of the user 16. The saddlebags 26a and 26b can also be supported by the waist band 56 and the support band 58. As shown, the saddlebags 26a and 26b are positioned against and around a respective leg of the user 16, and air vents 60a and 60b are provided respectively for saddlebags 26a and 26b to vent air as liquid is collected in the saddlebags 26a and 26b. For disclosure purposes only, combination conduits 24/46 are shown employed with the saddlebags 26a and 26b together, as envisioned for an alternate embodiment of the present invention. As disclosed above, depending on design requirements, the Tesla tube 46 may work in combination with the bladder 24 in various configurations. Further, FIG. 6 shows that a three-way valve 22 is positioned to be easily manipulated by the user 16 for purposes disclosed above.

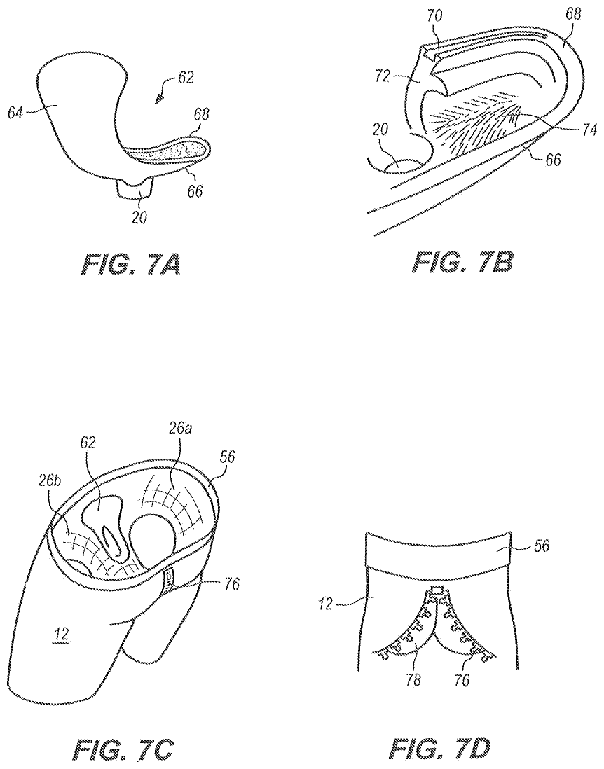

A female catcheter is shown in FIG. 7A and is generally designated 62. As shown, the female catcheter 62 is formed with a cup-shaped upper end 64 and a scoop-shaped lower end 66 that are mutually surrounded by a lip 68. The female catcheter 62 is preferably made of a soft silicone material and, as disclosed above, the upper end 64 and the lower end 66 are dimensioned for comfortable engagement of the lip 68 around the genital area of the user 16. In more detail, as shown in FIG. 7B, the lip 68 of the female catcheter 62 is formed with a groove 70 for holding a hydrocolloid which will create a liquid-tight seal between the female catcheter 62 and the user 16. At the lower end 66 of the female catcheter 62, a backsplash guard 72 and faux hair 74 are formed adjacent the lip 68 and around the one-way valve 20 in the lower end of the catcheter 62 to suppress fluid turbulence in the catcheter 62.

FIG. 7C shows the female catcheter 62 positioned and mounted in the outer garment layer 12 of the system 10 for disposal of liquid waste. FIG. 7C also shows that the outer garment layer 12 includes a zipper 76 which is included at the back of the outer garment layer 12. The purpose of zipper 76 is to expose the derriere 78 of the user 16, as shown in FIG. 7D, for the elimination of solid waste.

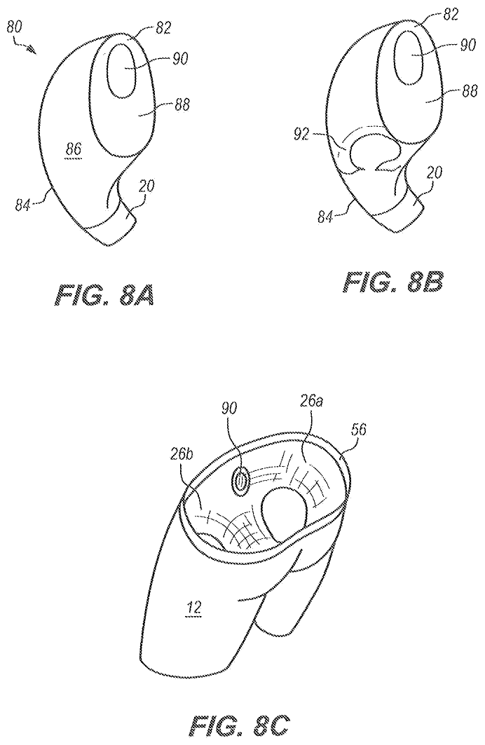

A male catcheter, generally designated 80, is shown in FIG. 8A. In FIG. 8A it is seen that the male catcheter 80 has an upper end 82 and a lower end 84, with a hollow collection chamber 86 formed therebetween. Structurally, the upper end 82 of the male catcheter 80 is formed with a contour surface 88 having a hole 90 for insertion of the penis of the male user 16. Also included is an anti-backflow ring 92 which is formed inside the collection chamber 86. As shown in FIG. 88, the anti-backflow ring 92 is located within the lower end 84 of the collection chamber 86 at a distance from the one-way valve 20.

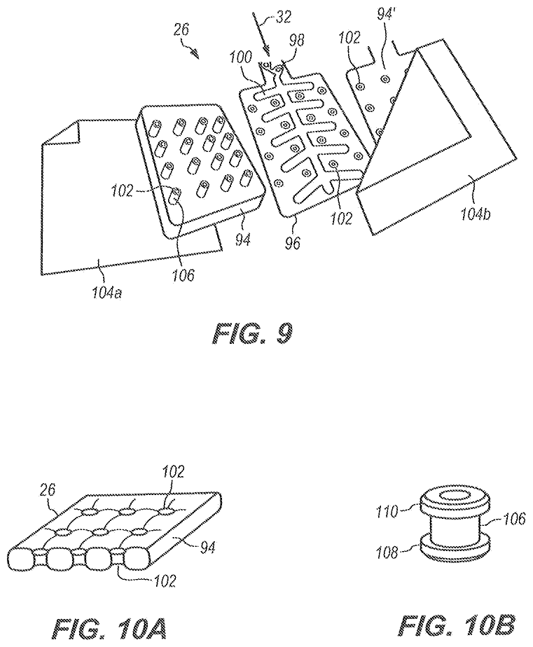

The construction of a saddlebag 26 is shown in FIG. 9. The essential element of the saddlebag 26 is an absorbent layer 94 of a material that is impregnated with a drying agent. Preferably, the material of the layer 94 is a compressed sponge and the drying agent is sodium polyacrylate. Further, a fluid channel complex 96 is provided that includes a fluid access port 98 that brings liquid on the pathway 32 (see FIG. 3) from the bladder 24 to a channel pattern 100 on layer 94 in the saddlebag 26. Thus, liquid in the system 10 is brought into direct contact with the absorbent material of layer 94.

As shown in FIG. 9, there are several possible configurations for the absorbent layer 94 in the saddlebag 26. For one, the complex channel pattern 100 can be formed directly on the layer 94, or it can be established as a separate template. Moreover, there may be a plurality of layers 94, such as is indicated by the additional layer 94′. In all configurations, the absorbent layer 94 is enclosed between a lower protective layer 104a and an upper protective layer 104b. Preferably, the protective layers 104a and 104b are made of a fire resistant material. A plurality of holes 102 are also provided for the saddlebag 26 to allow air to flow through the saddlebag 26.

A fabrication for assembly in the saddlebag 26 is shown in FIG. 10A, where grommets 106, such as shown in FIG. 108, establish holes 102 through the layer 94. Construction of the saddlebag 26 is then completed when a grommet 106 is positioned in a respective hole 102 and the lower end 108 of the grommet 106 is affixed to the lower protective layer 104a and the upper end 110 of the grommet 106 is affixed to the upper protective layer 104b.

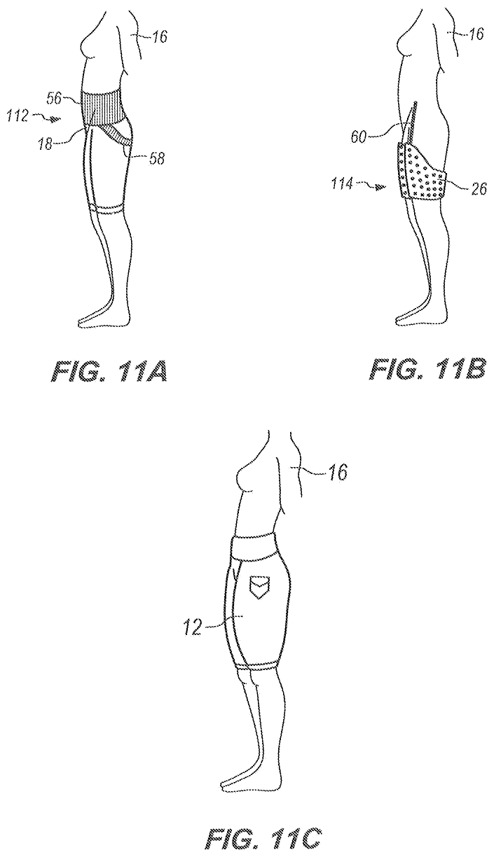

The steps involved in the assembly of a system 10 for operational purposes are sequentially shown in the FIGS. 11 A-C. In FIG. 11A, the innermost garment layer 112 is the component to which the fluid flow components are subsequently attached. In sum, the innermost garment layer 112 will include the catcheter 18, the waistband 56 and the support band 58. A middle garment layer 114 shown in FIG. 11B essentially includes the fluid flow components of the system 10. These components constitute the innermost garment layer 112 of the system 10 and basically include the saddlebags 26a and 26b with an incorporated bladder 24, Tesla tubes 46 and a three-way valve 22 (see FIG. 2). An important feature of the present invention is that both the innermost garment layer 112 and the middle garment layer 114 are incorporated into the outermost garment layer 12. This combination garment is shown in FIG. 11C, with the user 16 ready to don a flight suit (not shown).

While the particular Device for In-Flight Urine Elimination as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A disposable system for collecting fluid waste from a user which comprises:
a receiving unit defining a first end and a second end with a fluid collection chamber formed therebetween, wherein the first end of the collection chamber is adapted to conform with the body of the user, around the genitalia, to create a fluid tight seal therewith;
a tubular shaped bladder adapted to be connected in fluid communication with the collection chamber via the second end of the receiving unit;
at least one fluid waste collecting saddlebag connected with the bladder, and adapted to conform with the body of the user;
at least one Tesla tube connected between the collection chamber and the bladder for establishing a one-way fluid flow channel into the saddlebag;
a valve system connecting the collection chamber with the Tesla tube for controlling fluid flow from the receiving unit through the Tesla tube and into the bladder for subsequent distribution of fluid from the bladder and into a respective saddlebag;
a garment adapted to be worn by the user, wherein each saddlebag is incorporated into the garment for placement at a predetermined location on the user's body while the garment is being worn.

2. The system of claim 1 wherein each saddlebag comprises:
a layer of fluid absorbent material having an upper surface and a lower surface, wherein the layer is impregnated with a drying agent and wherein the upper surface is formed with a fluid channel complex having an access port for receiving fluid from the bladder and into the fluid channel complex, and wherein a plurality of holes is created between the upper and lower surfaces through the layer with the fluid channel complex being distanced from the holes;
a liquid proof bag for receiving the layer of absorbent material therein, wherein the bag is formed with a plurality of holes for respective alignment with the holes of the layer in the bag; and
a plurality of grommets, wherein each grommet is positioned in a respective hole with a first end connected with the bag in a liquid tight seal over the upper surface of the bag, and a second end connected with the bag in a liquid tight seal over the lower surface of the bag to establish air holes through the saddlebag.

3. The system of claim 2 wherein the absorbent material is compressed sponge and the drying agent is sodium polyacrylate and further wherein the garment is made of a fire resistant material.

4. The system of claim 1 wherein the tubular bladder is lengthwise bifurcated by a flexible membrane to create a fluid holding section and a pressure section along the length of the bladder, and wherein the system includes a fluid control unit which comprises:
a one-way directional flow valve connected between the collection chamber of the receiving unit and the Tesla tube for selectively controlling fluid flow into the fluid holding section of the bladder and from there into the saddlebag; and
a three-way stop cock valve connected between the pressure section of the bladder and a hand pump for inflating the pressure section with an overpressure to partially collapse the fluid holding section in the bladder, and for subsequently relieving the overpressure in the pressure section to deflate the pressure section and create a suction in the fluid holding section to draw fluid waste flow from the fluid chamber and into the saddlebag.

5. The system of claim 4 wherein the hand pump is temporarily replaced by a collection bulb and the three-way stop cock valve is reconfigured to establish direct fluid communication between the collection chamber of the receiving unit and the collection bulb to bypass the saddlebag and collect fluid waste in the collection bulb for immediate disposal.

6. The system of claim 5 wherein the flexible membrane is made of an elastomeric material.

7. The system of claim 1 wherein the receiving unit is a catcheter adapted for a female user, and wherein the catcheter comprises:
a soft silicone cup forming the fluid collection chamber and dimensioned to accommodate a fluid flow at least equal to 2.7 liters per minute;
a circumferential lip formed to encircle the first end of the collection chamber, wherein the lip is grooved to include a hydrocolloid for adhering the collection chamber to the body of the user;
a one-way valve mounted at the second end of the collection chamber; and
a backsplash guard, in combination with faux hair structures, formed adjacent the second end of the collection chamber.

8. The system of claim 7 further comprising a packet of coconut oil for use by the user when removing the collection chamber from the body.

9. The system of claim 8 further comprising a one-way valve connected to the second end of the collection chamber to prevent backflow of fluid into the collection chamber.

10. The system of claim 1 wherein the receiving unit is a catcheter adapted for a male user, and wherein the catcheter comprises;
a hollow collection chamber made of a soft silicone having a first end and a second end wherein the first end is formed as a contour surface for a conforming engagement of the contour surface with the body of the male user, and further, wherein the contour surface is formed with a hole for the insertion of a penis therethrough and into the collection chamber and dimensioned to accommodate a fluid flow at least equal to 2.7 liters per minute; and
an anti-flowback ring affixed inside the collection chamber adjacent the second end thereof for use in combination with a one-way valve affixed to the second end to reduce fluid flowback into the collection chamber.

11. A disposable system for collecting fluid waste from a user which comprises:
a garment worn by the user;
a means for receiving fluid waste from the user, wherein the receiving means is mounted on the garment;
a one-way valve connected in fluid communication with the receiving means to alternately select between an external fluid flow line from the receiving means to an external collection means for immediate disposal, and an internal fluid flow line from the receiving means along a pathway mounted on the garment, and wherein the one-way valve is mounted on the garment; and
a Tesla tube, wherein the Tesla tube establishes the internal fluid flow line and is mounted on the garment for receiving the internal fluid flow from the one-way valve for transfer to an internal collection means mounted on the garment and subsequent disposal with the garment.

12. The system of claim 11 wherein the receiving means is a catcheter which comprises a hollow fluid collection chamber defining a first end and a second end, wherein a lip is formed to encircle the first end of the hollow fluid collection chamber and is adapted to conform with the body of the user, around the genitalia, to create a fluid tight seal therewith.

13. The system of claim 12 further comprising a tubular shaped bladder, wherein the bladder is lengthwise bifurcated by a flexible membrane to create a pressure section and a fluid holding section along the length of the bladder and wherein the fluid holding section is connected in fluid communication with the second end of the Tesla tube.

14. The system of claim 13 further comprising a fluid control unit which comprises a three-way stop cock valve external to the garment for selectively establishing a first fluid connection line, a second fluid connection line, and a third fluid connection line: wherein the first fluid connection line connects the collection chamber in direct fluid communication with an external collection bulb to collect fluid waste for immediate disposal; wherein the second fluid connection line establishes direct fluid communication between the collection chamber of the receiving unit and the bladder to collect fluid waste for subsequent disposal with the garment; and wherein the third fluid connection line connects an external inflator with the pressure section of the bladder for first inflating the pressure section of the bladder with an overpressure to partially collapse the fluid holding section in the bladder and then subsequently relieving the overpressure in the pressure section to deflate the pressure section and create a suction in the fluid holding section to draw fluid waste flow from the fluid chamber and into the fluid holding section of the bladder.

15. The system of claim 14 wherein the internal collection means is a saddlebag comprising:
a layer of fluid absorbent material having an upper surface and a lower surface, wherein the layer is impregnated with a drying agent and wherein the upper surface is formed with a fluid channel complex with an access port for receiving fluid from the bladder and into the fluid channel complex, and wherein a plurality of holes is created between the upper and lower surfaces through the layer with the fluid channel complex being distanced from the holes;
a liquid proof bag for receiving the layer of absorbent material therein, wherein the bag is formed with a plurality of holes for respective alignment with the holes on the upper and lower surfaces of the layer in the bag; and
a plurality of grommets, wherein each grommet is positioned in a respective hole with a first end connected with the bag in a liquid tight seal over the upper surface of the bag, and a second end connected with the bag in a liquid tight seal over the lower surface of the bag to establish air holes through the saddlebag.

16. The system of claim 15 wherein the absorbent material is compressed sponge and the drying agent is sodium polyacrylate and further wherein the garment is made of a fire resistant material.

17. A method for manufacturing a disposable system for collecting fluid waste from a user which comprises the steps of:
forming a hollow collection chamber having a first end and a second end, wherein the first end is adapted to establish a fluid tight engagement around the genitalia of the user;
connecting a tubular shaped bladder in fluid communication with the second end of the collection chamber via an interconnecting Tesla one-way tube between the collection chamber and the bladder;
constructing a fluid waste collecting saddlebag connected in fluid communication with the bladder; and
incorporating the hollow collection chamber, the Tesla one-way tube, the bladder, and the saddlebag into a garment for wear by a user.

18. The method of claim 17 wherein the collection chamber is made of a soft silicone and is formed with a lip including a hydrocolloid material, and wherein the garment is made of a fire resistant material.

19. The method of claim 17 wherein the constructing step comprises the steps of:
impregnating a layer of an absorbent material with a drying agent, wherein the layer has an upper surface and a lower surface;
forming a plurality of holes between the upper and lower surfaces of the layer;
creating a fluid channel complex on the upper surface of the layer having an access port to the fluid channel complex, wherein the fluid channel complex is distanced from the holes;
providing a liquid proof bag for receiving the impregnated layer of absorbent material therein, wherein the bag is formed with a plurality of holes for respective alignment with the holes of the layer in the bag, and the layer is positioned to establish a fluid connection for the channel complex, via the access port to the bladder; and
inserting a plurality of grommets into a respective hole through the layer/bag combination with a first end of each grommet connected with the bag in a liquid tight seal over the upper surface of the bag, and a second end of each grommet connected with the bag in a liquid tight seal over the lower surface of the bag to establish air holes through the fluid waste collecting saddlebag.

20. The method of claim 19 further comprising the steps of:
bifurcating the bladder along the length of the tubular bladder with a flexible membrane to create a pressure section and a fluid holding section, wherein each section extends along the length of the bladder;
connecting a one-way directional flow valve between the collection chamber and the Tesla tube for directing fluid waste from the fluid chamber, via the Tesla tube and the fluid holding section of the bladder for collection in the saddlebag;
connecting a three-way stop cock valve between the pressure section of the bladder and a hand pump via the second fluid connection for inflating the pressure section with an overpressure to partially collapse the fluid holding section in the bladder, and for relieving the overpressure in the pressure section to deflate the pressure section and create a suction in the fluid holding section to facilitate fluid waste flow from the fluid chamber of the receiving unit via the fluid holding section and into the saddlebag; and
adapting the three-way stop cock valve for selective connection, via the first fluid connection, between the fluid chamber and a collection bulb, to bypass the second fluid connection.
